(19) United States
(12) Patent Application Publication
(10) Pub. No.: US 2021/0198689 A1
(43) Pub. Date: Jul. 1, 2021

(54) Multimodal Vector for Dendritic Cell Infection

(71) Applicants: Nant Holdings IP, LLC, Culver City, CA (US); NantCell, Inc., Culver City, CA (US)

(72) Inventors: Patrick Soon-Shiong, Culver City, CA (US); Kayvan Niazi, Culver City, CA (US); Shahrooz Rabizadeh, Culver City, CA (US)

(21) Appl. No.: 16/081,014

(22) PCT Filed: Mar. 20, 2017
(86) PCT No.: PCT/US2017/023117

§ 371 Date: Aug. 29, 2018

(51) Int. Cl. C12N 15/86; C07K 14/705

(52) U.S. Cl. CPC C12N 15/86; C07K 14/70525; C12N 2710/10343; C07K 14/70553; C12N 2710/10041; C07K 14/70532

(57) ABSTRACT

Recombinant viruses and viral nucleic acids are contemplated that provide to the infected cell various regulatory molecules that stimulate T-cell and NK-cell activity and that suppress inhibition of T-cell and NK-cell activity. Most preferably, the virus and viral nucleic acid will further include a human cancer-associated sequence, and especially a sequence that encodes a plurality of cancer associated antigens, cancer specific antigens, and/or patient and tumor specific neoantigens. Especially preferred regulatory molecules include CD80 (B7.1), CD86 (B7.2), CD54 (ICAM-1/BB2), CD11 (LFA-1), and an inhibitor of CTLA-4.

This application claims priority to U.S. provisional application Ser. No. 62/310,551, filed Mar. 18, 2016 and claims priority to U.S. provisional application Ser. No. 62/313,596, filed Mar. 25, 2016.

### FIELD OF THE INVENTION

The field of the invention is recombinant nucleic acid vectors, particularly adenovirus vectors for cell transfection with at least dual function.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Recent advances in immune therapy for cancer has yielded significant improvements in treatment outcome. For example, increased capability to characterize cancer cells on a molecular level has allowed for more targeted treatments. Among other targets, immune therapy has made use of cancer associated antigens (e.g., CEA-1), cancer specific antigens (e.g., HER2), or patient- and tumor-specific neoepitopes in an attempt to direct genetically altered immune competent cells to the cancer.

However, with the increasing experience in modulating activity of immune competent cells, the vast complexity of regulatory processes required to generate a therapeutically effective immune response has become evident. For example, depending on the type of cancer related antigens, some antigens will not or only insufficiently be presented by the MHC-I and MHC-II system of a patient. In another example, tumors frequently generate a microenvironment that down-regulates activity of cells otherwise cytotoxic to cancer cells. Additionally, costimulatory signals are often required to promote a robust immune response, but are not always present or present in sufficient quantities. Therefore, while production of genetically modified immune competent cells (e.g., CAR-T) is often relatively simple, their effectiveness in vivo is often reduced by factors not readily compensated for. Among other difficulties, proper antigen presentation, activation, and reduction of suppressing signals often interfere with a proper immune response.

Effective stimulation of T cells is thought to require formation of a durable immune synapse that involves a well choreographed assembly of numerous proteins (Science (1999) 285 (5425): 221-227; Science (2002) 295 (5559): 1539-1542). In an attempt to simulate the formation of an immune synapse, various signaling molecules for stimulating T cells were fixed onto a carrier in a pre-oriented fashion with respect to spacing, distribution, and pattern as described in US 2008/0317724. Notably, the inventors observed that T cell activation in such systems required specific spatial arrangements of CD28 and T cell receptors. However, various other factors and cell-cell interaction between an antigen presenting cell and T cells were not present and signaling and activation may therefore be less than effective in vivo.

In further known methods of T cell activation, co-expression of secreted antigen and selected costimulatory molecules in cells was reported in WO 2016/127015. However, as the costimulatory molecules were secreted fusion proteins and as the antigen was also secreted and not matched to a specific HLA type, proper antigen presentation was likely not ensured in the context of the costimulatory molecules.

Expression of certain costimulatory molecules (B7-1/ICAM-1/LFA-3) and cancer or tumor associated antigen from a poxviral vector was reported to activate CD8⁺ and CD4⁺ cells, but failed to increase apoptosis relative to comparable systems that expressed B7-1 only (Cancer Research (1999) Vol 59, 5800-5807; Biomedicines (2016), Vol 4, 19). The antigen in these systems was CEA, and it should be noted that not all CEA fragments are presented equally by different HLA types. Moreover, as CEA is also expressed in normal non-cancer cells, autoimmune reactions cannot be ruled out possible. Moreover, the viruses employed in these studies was immunogenic and so allowed only single administration.

In yet another approach, OX40 (CD134) with an agonist anti-OX40 mAb enhanced antitumor immunity by augmenting T cell differentiation and systemic antibody mediated blockade of the checkpoint inhibitor CTLA-4 (Cancer Immunol Res (2014) Vol 2(2): 142-153). Notably, combined anti-OX40/anti-CTLA-4 immunotherapy did significantly enhance tumor regression and survival of tumor-bearing hosts in a CD4 and CD8 T cell-dependent manner. However, systemic anti-CTLA-4 immunotherapy has been associated with a higher risk of cytokine storm. In a similar approach, vaccination targeting a tumor-associated antigen toward crosspresenting dendritic cells was combined with antiOX40/antiCTLA-4 immunotherapy (Journal for ImmunoTherapy of Cancer (2016) 4:31). Unfortunately, while promising results were indeed achieved, the development of a protective immune response requires a substantially intact immune system that is in many patients no longer available (e.g., due to repeated chemotherapy and/or radiation).

In addition, many cancer vaccines that are delivered using viral vehicles tend to be ineffective in eliciting an immune response against the antigenic cargo due to the host response against the viral vector and as such often reduce the chances to deliver the DNA payload to produce cancer epitopes that are designed to give rise to an immune response against the tumor. Consequently, administration of the viral vaccine is generally limited to a single attempt. Moreover, as the recombinant DNA is transcribed and translated, the resulting products tend to favor an immune reaction via the MHC-I system. However, effective immunotherapy also requires a robust T-cell and NK cell response, which is generally stimulated by “Type I” CD4+ T cells which are activated by the MHC-II system.

Therefore, even though numerous methods and compositions are known in the art to generate an anti-tumor immune response, all or almost all of them suffer from one or more disadvantages. Consequently, there remains a need for improved compositions and methods for immunotherapy of cancer.

### SUMMARY OF THE INVENTION

The inventive subject matter is directed to compositions and methods in which a recombinant (preferably replication deficient and non-immunogenic) virus or recombinant viral nucleic acid encodes a plurality of stimulatory molecules, an inhibitor of an immune checkpoint receptor, and one or more human cancer-associated sequences to so help elicit a durable and therapeutically effective immune response upon administration of the virus to a person in need thereof. Most typically, the virus will be administered to the patient to infect dendritic cells that then interact with CD8⁺ and CD4⁺ T-cells to produce robust immune response and generate immune memory. In addition to only using neoepitopes as targets for immune therapy, dual-mode administration (and especially via recombinant expression and injection) of stimulators and/or inhibitors of immune suppression are thought to even further enhance efficacy of such therapies.

In one aspect of the inventive subject matter, the inventors contemplate a recombinant nucleic acid vector that comprises at least a portion of a viral genome that includes a recombinant sequence portion encoding a plurality of genes, wherein the recombinant sequence portion is operably coupled to a regulatory sequence to allow for expression of the plurality of genes. Most typically, the plurality of genes encode four distinct stimulatory molecules and at least one (preferably membrane anchored) inhibitory ligand for an immune checkpoint receptor, and the viral genome has at least one mutated or deleted protein coding sequence to so reduce immunogenicity of the virus encoded by the viral genome.

With respect to the four distinct stimulatory molecules it is generally preferred that the stimulatory molecules include at least one, or at least two, or at least three, or all of CD80 (B7.1), CD86 (B7.2), CD54 (ICAM-1/BB2), and CD11 (LFA-1). Preferred immune checkpoint receptors include CTLA-4 or PD-1, and it is generally contemplated that the inhibitory ligand will comprise at least one transmembrane domain that anchors the ligand to a cell membrane. Moreover, it is generally preferred that the recombinant sequence portion further comprises one or more human cancer-associated sequences (e.g., cancer associated antigen, a cancer specific antigen, and a patient- and tumor-specific neoantigen). Where desired, the human cancer-associated sequence will further comprise a trafficking sequence that preferentially directs a gene product encoded by the cancer-associated sequence to the cytoplasmic compartment or the lysosomal or endosomal compartment of a cell hosting the recombinant nucleic acid vector. Additionally, it is preferred that the virus is replication deficient and/or an adenovirus, and that the mutated or deleted protein coding sequence is E1, E2b, and/or E3 of adenovirus type 5.

Therefore, the inventors also contemplate a virus comprising the recombinant nucleic acid vector as presented above. Most preferably, the virus is a recombination deficient adenovirus lacking the E2b gene, and the distinct stimulatory molecules are one or more of CD80 (B7.1), CD86 (B7.2), CD54 (ICAM-1/BB2), and CD11 (LFA-1), wherein the immune checkpoint receptor is CTLA-4, and wherein the recombinant sequence portion further comprises a human cancer-associated sequence.

Such recombinant nucleic acids and viruses are particularly deemed to infect an antigen presenting cell to thereby stimulate T cell activation in a T cell that contacts the antigen presenting cell. Therefore, the inventors also contemplate a method of stimulating an immune response in a mammal that comprises a step of administering the virus (e.g., by subcutaneous or subdermal injection) under a protocol effective to stimulate the immune response. Where desired, such methods will further include administering low-dose chemotherapy or low-dose radiation therapy to the mammal, preferably in metronomical fashion.

Various objects, features, aspects and advantages of the inventive subject matter will become more apparent from the following detailed description of preferred embodiments.

### DETAILED DESCRIPTION

The inventors have discovered that immune therapeutic compositions can be prepared using a viral vector, and most preferably an adenoviral vector, that includes a recombinant nucleic acid encoding a plurality of (co-)stimulatory molecules and at least one inhibitor of an immune checkpoint receptor that is preferably anchored to a cell membrane of an antigen presenting cell. Moreover, such recombinant virus or viral vector will further include one or more human cancer-associated sequences to stimulate an immune reaction against cells presenting proteins encoded by the cancer-associated sequences. Thus, an antigen presenting cell expressing the recombinant proteins will therefore present the antigen in the context of both stimulatory factors and anti-inhibitory factors that promote sufficient interaction for an antigen specific T cell activation.

It is still further preferred that the virus is non-immunogenic (i.e., can be administered at least two, at least three, at least four or even more times without eliciting a protective immune response against the virus), replication deficient, and administered subcutaneously or subdermally to the patient to thereby preferentially infect dendritic cells. In one particularly preferred example, the viral vector is a recombinant adenovirus that has the E1, E2b, and E3 viral genes deleted to so reduce immunogenicity and increase capacity of payload. Introduced into such modified viral genome is then one or more expression cassettes that encode under suitable control elements (typically a constitutively active promoter) the co-stimulatory molecules are CD80 (B7.1) and CD86 (B7.2), activator molecules CD54 (ICAM-1/BB2) and CD11 (LFA-1), and an inhibitor for the immune checkpoint receptor CTLA-4 (e.g., a scFv, optionally with transmembrane domain). Also encoded in the recombinant nucleic acid are a plurality of cancer-associated sequences that are co-expressed with the stimulatory molecules and the inhibitory ligand. While not necessary, it is typically preferred that at least some of the cancer-associated sequences are directed to MHC-I processing pathways and/or MHC-II processing pathways by use of appropriate trafficking sequences.

Therefore, it should be appreciated that the virus (or viral vector) design presented herein will provide multiple benefits for triggering a strong and durable immune response against the cancer-associated sequences. First, and upon infection of a dendritic cell with the recombinant virus, the cancer-associated sequences are expressed and presented using MHC-I and/or MHC-II presentation pathways, which will increase the likelihood of producing appropriately activated CD4⁺ and CD8⁺ cells, which in turn is believed to increase the likelihood of proper antibody production and suitable T- and B-cell memory. In addition, as the cancer-associated sequences are preferably and coordinately expressed with various co-stimulatory molecules (and most preferably with CD80, CD86, CD54, and CD11) T-cell activation by such infected cells is increased as these cells present the MHC-bound epitopes together with co-stimulatory molecules. Additionally, potential inhibitory signaling is reduced by such infected cells as these cells also express an inhibitory ligand (typically membrane-bound) to CTLA-4 and/or PD-1 on CD4⁺ and CD8⁺ cells upon activation.

Viewed from another perspective, it should be appreciated that the viruses and viral vector constructs contemplated herein provide optimized activation to and suppress inhibition of CD4⁺ and CD8⁺ cells in the context of the presented cancer-associated sequences, which is thought to produce a robust and therapeutically effective immune response against cancer cells presenting the cancer-associated sequences. Such advantages are particularly beneficial where the virus is administered subcutaneously or subdermally to increase infection of dendritic cells, which in turn activate in an epitope specific manner immune competent cells, and especially CD4⁺ T-cells, CD8⁺ T-cells, and NK cells.

However, it should be appreciated suitable viral vectors (and with that viral nucleic acid vectors) need not be limited to adenoviruses as described above, and it should be recognized that the particular choice of vector is not critical to the inventive subject matter. Therefore, suitable viruses include adenoviruses, adeno-associated viruses, alphaviruses, herpes viruses, lentiviruses, etc. However, adenoviruses are particularly preferred. Moreover, it is further preferred that the virus is a replication deficient and non-immunogenic virus, which is typically accomplished by targeted deletion of selected viral proteins (e.g., E1, E3 proteins for adenovirus). Such desirable properties may be further enhanced by deleting the E2b gene function.

Where the virus is replication deficient, it should be recognized that viral cultures can be prepared using cells lines that provide the lacking function (e.g., polymerase gene). For example, relatively high titers of recombinant viruses can be achieved using genetically modified human 293 cells as has been recently reported (e.g., J Virol. 1998 February; 72(2): 926-933). Further particularly preferred aspects of suitable virus constructs are described in U.S. Pat. Nos. 6,083,750, 6,063,622, 6,057,158, 6,451,596, 7,820,441, 8,298,549, and 8,637,313. Most typically, and as already addressed above, the desired nucleic acid sequences for expression from virus infected cells are under the control of appropriate regulatory elements well known in the art. Modification of viral genomes or viral vectors will generally follow standard procedures that are well known in the art (see e.g., Gene Therapy by Mauro Giacca, Springer Science & Business Media, Nov. 1, 2010. Or A Guide To Human Gene Therapy by Roland Herzog (Ph. D.), Sergei Zolotukhin; World Scientific, 2010. Or Gene Therapy Protocols by Paul D. Robbins, Humana Press, 1997).

With respect to stimulating molecules, it is generally contemplated that co-stimulatory molecules as well as other stimulating molecules are deemed suitable for use herein, as well as their corresponding muteins, truncated, and chimeric forms. For example, especially suitable co-stimulatory molecules include CD80, CD86, CD40, ICOS-L, B7-H3, B7-H4, CD70, OX40L, 4-1BBL, while other stimulatory molecules with less defined (or understood) mechanism of action include GITR-L, TIM-3, TIM-4, CD48, CD58, ICAM-1, LFA3, and members of the SLAM family. However, especially preferred molecules for coordinated expression with the cancer-associated sequences include CD80 (B7-1), CD86 (B7-2), CD54 (ICAM-1) and CD11 (LFA-1). Sequences for contemplated stimulatory molecules are known in the art, and all of the sequences (RNA as well as cDNA and genomic DNA) are deemed suitable for use herein.

Likewise, there are several inhibitory signal pathways known for T-cell activation, and all compounds reducing inhibition of T-cell activation are contemplated herein. For example, peptide molecules are contemplated that bind to or otherwise inhibit signaling through PD-1, PD1H, TIM1 receptor, 2B4, CTLA-4, BTLA, and CD160. Such binding or other inhibition may be triggered by expression and secretion of suitable antagonistic ligands or binding fragments (e.g., scFv), and/or may be mediated by expression and membrane bound presentation. Therefore, contemplated inhibitory ligands may also comprise a transmembrane domain fused to the peptide ligand. There are numerous transmembrane domains known in the art, and all of those are deemed suitable for use herein, including those having a single alpha helix, multiple alpha helices, alpha/beta barrels, etc. For example, contemplated transmembrane domains can comprise comprises the transmembrane region(s) of the alpha, beta, or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, or PAG/Cbp. Where a fusion protein is desired, it is contemplated that the recombinant chimeric gene has a first portion that encodes the transmembrane region(s), wherein the first portion is cloned in frame with a second portion that encodes the inhibitory protein.

It should be appreciated that all of the above noted stimulatory genes and genes coding for inhibitory proteins that interfere with/down-regulate checkpoint inhibition are well known in the art, and sequence information of these genes, isoforms, and variants can be retrieved from various public resources, including sequence data bases accessible at the NCBI, EMBL, GenBank, RefSeq, etc. Moreover, while the above exemplary stimulating molecules are preferably expressed in full length form as expressed in human, modified and non-human forms are also deemed suitable so long as such forms assist in stimulating or activating T-cells. Therefore, muteins, truncated forms and chimeric forms are expressly contemplated herein.

With respect to the cancer-associated sequences it should be appreciated that any epitope that is cancer associated, specific to a type of cancer, or a patient-specific neoepitope is suitable for use herein, particularly where the epitope is expressed (preferably above healthy control), and where the expressed epitopes are also proven or predicted to bind to the respective binding motifs of the MHC-I and/or MHC-II complex.

For example, neoepitopes may be identified from a patient tumor in a first step by whole genome analysis of a tumor biopsy (or lymph biopsy or biopsy of a metastatic site) and matched normal tissue (i.e., non-diseased tissue from the same patient) via synchronous comparison of the so obtained omics information. So identified neoepitopes can then be further filtered for a match to the patient's HLA type to increase likelihood of antigen presentation of the neoepitope. Most preferably, and as further discussed below, such matching can be done in silico. Most typically, the patient-specific epitopes are unique to the patient, but may also in at least some cases include tumor type-specific neoepitopes (e.g., Her-2, PSA, brachyury) or cancer-associated neoepitopes (e.g., CEA, MUC-1, CYPB1). Thus, it should be appreciated that the adenoviral nucleic acid construct (or nucleic acid construct for other delivery) will include a recombinant segment that encodes at least one patient-specific neoepitope, and more typically encode at least two or three more neoepitopes and/or tumor type-specific neoepitopes and/or cancer-associated neoepitopes. Where the number of desirable neoepitopes is larger than the viral capacity for recombinant nucleic acids, multiple and distinct neoepitopes may be delivered via multiple and distinct recombinant viruses.

With respect to the step of obtaining omics information from the patient to identify one or more neoepitopes it is contemplated that the omics data are obtained from patient biopsy samples following standard tissue processing protocol and sequencing protocols. While not limiting to the inventive subject matter, it is typically preferred that the data are patient matched tumor data (e.g., tumor versus same patient normal), and that the data format is in SAM, BAM, GAR, or VCF format. However, non-matched or matched versus other reference (e.g., prior same patient normal or prior same patient tumor, or homo statisticus) are also deemed suitable for use herein. Therefore, the omics data may be ‘fresh’ omics data or omics data that were obtained from a prior procedure (or even different patient).

Regardless of the nature of the reference sequence (e.g., matched normal), it is generally preferred that the reference sequence is used to calculate a plurality of epitopes. Most typically, the epitopes will be calculated to have a length of between 2-50 amino acids, more typically between 5-30 amino acids, and most typically between 9-15 amino acids, with a changed amino acid preferably centrally located or otherwise situated in a manner that improves its binding to MHC. For example, where the epitope is to be presented by the MHC-I complex, a typical epitope length will be about 8-11 amino acids, while the typical epitope length for presentation via MHC-II complex will have a length of about 13-17 amino acids. It is still further preferred that the so calculated epitopes and neoepitopes are then analyzed in silico for their affinity to the patient-specific HLA-type (MHC-I and MHC-II) as further described below in more detail. It should be appreciated that knowledge of HLA affinity for such neoepitopes provides at least two items of valuable information: (a) deletion of an epitope otherwise suitable for immunotherapy can be recognized and immunotherapy be adjusted accordingly so as to not target the deleted epitope, and (b) generation of a neoepitope suitable for immunotherapy can be recognized and immunotherapy be adjusted accordingly so as to target the neoepitope.

Moreover, and as further described below, it should be appreciated that the choice of neoepitope is also further guided by investigation of expression levels and sub-cellular location of the neoepitope. For example, where the neoepitope is not or only weakly expressed relative to matched normal (e.g., equal or less than 20% of matched normal expression), the neoepitope may be eliminated from the choice of suitable neoepitopes. Likewise, where the neoepitope is identified as a nuclear protein, the neoepitope may be eliminated from the choice of suitable neoepitopes. On the other hand, positive selection for neoepitopes may require partially extracellular or transmembrane presence of the neoepitope and/or an expression level of at least 50% as compared to matched normal. Expression levels can be measured in numerous manners known in the art, and suitable manners include qPCR, qLCR, and other quantitative hybridization techniques.

It is generally contemplated that genomic analysis can be performed by any number of analytic methods, however, especially preferred analytic methods include WGS (whole genome sequencing) and exome sequencing of both tumor and matched normal sample. Likewise, the computational analysis of the sequence data may be performed in numerous manners. In most preferred methods, however, analysis is performed in silico by location-guided synchronous alignment of tumor and normal samples as, for example, disclosed in US 2012/0059670A1 and US 2012/0066001A1 using BAM files and BAM servers.

So identified and selected neoepitopes can then be further filtered in silico against an identified patient HLA-type. Such HLA-matching is thought to ensure strong binding of the neoepitopes to the MHC-I complex of nucleated cells and the MHC-II complex of specific antigen presenting cells. Targeting both antigen presentation systems is particularly thought to produce a therapeutically effective and durable immune response involving both, the cellular and the humoral branch of the immune system. HLA determination for both MHC-I and MHC-II can be done using various methods in wet-chemistry that are well known in the art, and all of these methods are deemed suitable for use herein. However, in especially preferred methods, the HLA-type can also be predicted from omics data in silico using a reference sequence containing most or all of the known and/or common HLA-types as is shown in more detail below. In short, a patient's HLA-type is ascertained (using wet chemistry or in silico determination), and a structural solution for the HLA-type is calculated or obtained from a database, which is then used as a docking model in silico to determine binding affinity of the neoepitope to the HLA structural solution. Suitable systems for determination of binding affinities include the NetMHC platform (see e.g., Nucleic Acids Res. 2008 Jul. 1; 36 (Web Server issue): W509-W512.), HLAMatchmaker (See URL www.epitopes.net/downloads.html), and IEDB Analysis Resource (See URL tools.immuneepitope.org/mhcii/). Neoepitopes with high affinity (e.g., less than 100 nM, less than 75 nM, less than 50 nM for MHC-I; less than 500 nM, less than 300 nM, less than 100 nM for MHC-I) against the previously determined HLA-type are then selected. In calculating the highest affinity, modifications to the neoepitopes may be implemented by adding N- and/or C-terminal modifications to the epitope to further increase binding of the virally expressed neoepitope to the HLA-type. Thus, neoepitopes may be native as identified or further modified to better match a particular HLA-type. Further aspects and considerations of HLA-matched neoepitopes are disclosed in US 2017/0028044, which is incorporated by reference herein.

With respect to routing the so identified and expressed neoepitopes to the desired MHC-system, it should be appreciated that the MHC-I presented peptides will typically arise from the cytoplasm via proteasome processing and delivery through the endoplasmatic reticulum. Thus, expression of the epitopes intended for MHC-I presentation will generally be directed to the cytoplasm as is further discussed in more detail below. On the other hand, MHC-II presented peptides will typically arise from the endosomal and lysosomal compartment via degradation and processing by acidic proteases (e.g., legumain, cathepsin L and cathepsin S) prior to delivery to the cell membrane. Thus, expression of the epitopes intended for MHC-II presentation will generally be directed to the endosomal and lysosomal compartment as is also discussed in more detail below.

In most preferred aspects, signal peptides may be used for trafficking to the endosomal and lysosomal compartment, or for retention in the cytoplasmic space. For example, where the peptide is to be exported to the endosomal and lysosomal compartment targeting presequences and the internal targeting peptides can be employed. The presequences of the targeting peptide are preferably added to the N-terminus and comprise between 6-136 basic and hydrophobic amino acids. In case of peroxisomal targeting, the targeting sequence may be at the C-terminus. Other signals (e.g., signal patches) may be used and include sequence elements that are separate in the peptide sequence and become functional upon proper peptide folding. In addition, protein modifications like glycosylations can induce targeting.

Among other suitable targeting signals, the inventors contemplate peroxisome targeting signal 1 (PTS1), a C-terminal tripeptide, and peroxisome targeting signal 2 (PTS2), which is a nonapeptide located near the N-terminus. In addition, sorting of proteins to endosomes and lysosomes may also be mediated by signals within the cytosolic domains of the proteins, typically comprising short, linear sequences. Some signals are referred to as tyrosine-based sorting signals and conform to the NPXY or YXXØ consensus motifs. Other signals known as dileucine-based signals fit [DE]XXXL[LI] or DXXLL consensus motifs. All of these signals are recognized by components of protein coats peripherally associated with the cytosolic face of membranes. YXXØ and [DE]XXXL[LI] signals are recognized with characteristic fine specificity by the adaptor protein (AP) complexes AP-1, AP-2, AP-3, and AP-4, whereas DXXLL signals are recognized by another family of adaptors known as GGAs. Also FYVE domain can be added, which has been associated with vacuolar protein sorting and endosome function. In still further aspects, endosomal compartments can also be targeted using human CD1 tail sequences (see e.g., Immunology, 122, 522-531).

Trafficking to or retention in the cytosolic compartment may not necessarily require one or more specific sequence elements. However, in at least some aspects, N- or C-terminal cytoplasmic retention signals may be added, including a membrane-anchored protein or a membrane anchor domain of a membrane-anchored protein. For example, membrane-anchored proteins include SNAP-25, syntaxin, synaptoprevin, synaptotagmin, vesicle associated membrane proteins (VAMPs), synaptic vesicle glycoproteins (SV2), high affinity choline transporters, neurexins, voltage-gated calcium channels, acetylcholinesterase, and NOTCH. Thus, it should be appreciated that peptides can be routed to specific cellular compartments to so achieve preferential or even specific presentation via MHC-I or MHC-II.

Additionally, or alternatively, it should also be appreciated that one or more neoepitopes may be encoded by the recombinant nucleic acid for expression in a cell such that the neoepitope is presented at or on the surface of the cell for antibody recognition without complexation by MHC-I and/or MHC-II. Such approach may be performed in combination with MHC-I and/or MHC-II targeted presentation, or less preferably also alone. Viewed form a different perspective, it should be appreciated that the purpose of including such neo-epitopes is to generate antibodies that could work alone or in combination with the classic MHC presented peptide epitopes to augment the immune response against a target set of proteins (although the same mutated protein could in principle be expressed on the surface while its patient specific epitopes get shunted to the various MHC I or II compartments). Such surface presentation will be performed using chimeric proteins in which the peptide epitope is fused to a transmembrane sequence, and suitable transmembrane sequences include those discussed above. For further aspects and contemplations related to differential presentation of neoepitopes are disclosed in co-owned pending U.S. provisional application 62/466,846, which is incorporated by reference herein.

It should be further appreciated that the stimulating and inhibitory ligand for an immune checkpoint receptor may be expressed under control of the same promoter, and/or have individual or common promoter elements. Likewise, it is preferred that the expression of the human cancer-associated sequences is also contemporaneous with the expression of the regulatory molecules, and will therefore be most preferably under the same control (or same independent promoter sequences).

For example, it is generally preferred that all of the recombinant genes are expressed from a constitutive strong promoter (e.g., SV40, CMV, UBC, EF1A, PGK, CAGG promoter), however various inducible promoters are also deemed suitable for use herein. For example, contemplated inducible promoters include the tetracycline-inducible promoter, the myxovirus resistance 1 (Mx1) promoter, etc. In still other examples, and especially where the antigen presenting cells are expected to be in a tumor microenvironment, inducible promoters include those sensitive to hypoxia and promoters that are sensitive to TGF-β or IL-8 (e.g., via TRAF, JNK, Erk, or other responsive elements promoter). Moreover, promoters that are natively found with the respective recombinant genes are also contemplated.

Most typically, but not necessarily, all recombinant genes are co-expressed from the same promoter and so generate a single transcript, for example, with an internal ribosome entry (IRES) site, or may be transcribed from one or more separate promoters as respective single gene transcripts, or as tandem minigenes, or any other arrangement suitable for expression. In still further contemplated aspects, it should be appreciated that the recombinant nucleic acid may encoding the stimulatory molecules and the inhibitory ligand for an immune checkpoint receptor may be based on the respective known mRNA or cDNA sequences (and as such will not have introns), or may have artificial introns or may be based on the genomic sequence (and as such will have introns and exons with associated splice sites). Therefore, it is contemplated that a transcript from contemplated recombinant nucleic acids will include an IRES (internal ribosome entry site) or a 2A sequence (cleavable 2A-like peptide sequence) to allow for coordinated expression of the co-stimulatory molecules and other proteins.

It should also be noted that the recombinant nucleic acids may be administered as DNA vaccine, but it is generally preferred that the recombinant nucleic acid is part of a viral genome. The so genetically modified virus can then be used as is well known in gene therapy. Thus, with respect to recombinant viruses it is contemplated that all known manners of making recombinant viruses are deemed suitable for use herein, however, especially preferred viruses are those already established in therapy, including adenoviruses, adeno-associated viruses, alphaviruses, herpes viruses, lentiviruses, etc. Among other appropriate choices, adenoviruses are particularly preferred.

Moreover, it is further generally preferred that the virus is a replication deficient and non-immunogenic virus, which is typically accomplished by targeted deletion of selected viral proteins (e.g., E1, E3 proteins). Such desirable properties may be further enhanced by deleting E2b gene function, and high titers of recombinant viruses can be achieved using genetically modified human 293 cells as has been recently reported (e.g., J Virol. 1998 February; 72(2): 926-933). Most typically, the desired nucleic acid sequences (for expression from virus infected cells) are under the control of appropriate regulatory elements well known in the art.

So produced recombinant viruses may then be individually or in combination used as a therapeutic vaccine in a pharmaceutical composition, typically formulated as a sterile injectable composition with a virus titer of between 10⁴-10¹¹ virus particles per dosage unit. However, alternative formulations are also deemed suitable for use herein, and all known routes and modes of administration are contemplated herein. As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.). Most preferably, the recombinant virus is administered via subcutaneous or subdermal injection. However, in other contemplated aspects, administration may also be intravenous injection. Alternatively, or additionally, antigen presenting cells may be isolated or grown from cells of the patient, infected in vitro, and then transfused to the patient. Therefore, it should be appreciated that contemplated systems and methods can be considered a complete drug discovery system (e.g., drug discovery, treatment protocol, validation, etc.) for highly personalized cancer treatment.

In addition, it is contemplated that prophylactic or therapeutic administration of the viral vector may be accompanied by co-administration with immune checkpoint inhibitors and/or immune stimulatory compounds to reduce possible inhibitory action on T-cells. For example, especially preferred check point inhibitors include currently available inhibitors (e.g., pembrolizumab, nivolumab, ipilimumab), typically under the same protocol and dosage as commonly prescribed. It is also contemplated that checkpoint inhibition be accomplished by delivering inhibitory ligands/biologics genetically through inclusion on the plasmid/viral DNAs. Likewise, genetically modified NK cells may be administered to the patient concurrent with or before or after administration of the recombinant virus contemplated herein.

Yet further additional treatments in conjunction with administration of modified viruses contemplated herein include interleukin-type stimulatory molecules that may be encoded within the viral vector or administered separately as protein drug. For example, suitable stimulatory compounds include IL-2, IL-15, IL-21, etc, and the N72D mutant form of IL-15 or an IL-15 superagonist (e.g., ALT803) is especially preferred. Furthermore, treatment may be assisted by administering therapeutically effective antibodies to increase antibody-dependent cell-mediated cytotoxicity. Such antibodies may target cell- and patient specific neoepitopes (e.g., those identified as described above), cancer-specific antigens (e.g., PSA, PSMA, HER2, etc.), and/or cancer-associated antigens (e.g., targeting MUCSAC variants (e.g., ensituximab), CEACAM variants, etc.).

Therefore, in an exemplary method it is contemplated that the recombinant nucleic acid may be administered via subcutaneous or subdermal injection to preferably target dendritic cells, while the stimulatory and/or anti-inhibitory compositions may be separately injected (e.g., preferably via intratumoral injection, or subcutaneous or subdermal injection) to promote a local and/or systemic increase in immune response to the virally induced challenge. For example, stimulatory compositions will preferably include IL-15, IL-2, IL-17, and/or IL-21, and especially preferred IL-15 compositions will include an IL-15 superagonist (e.g., N72D mutant, which enhances binding of IL-15 to IL-2Rβγ), and preferred anti-inhibitory compositions include ipilimumab (Yervoy®), pembrolizumab (Keytruda®), and nivolumab (Opdivo®). Most typically, but not necessarily, the stimulatory and/or anti-inhibitory compositions are administered at dosages at or below the dosages approved or commonly employed, and in some aspects of the inventive subject matter, administration will be at a low-dose regimen (e.g., between 80-95%, between 60-85%, between 40-60%, between 20-40% or between 1-20% of standard, approved, or recommended dose).

Viewed from a different perspective, it should therefore be appreciated that contemplated systems and methods will comprise a patient and cancer specific component that is typically delivered via a recombinant nucleic acid (e.g., via viral vector) to so stimulate presentation of HLA-bound neoepitope, wherein the neoepitopes are presented in the context of at least one of a co-stimulatory molecule and an immune checkpoint inhibitor. Of course, it should also be recognized that suitable nucleic acid vectors may also include bacterial vectors, yeast vectors and yeast artificial chromosomes, as well as viral vectors. In addition, contemplated systems and methods will also comprise an immune stimulating component that is independently administered with respect to the neoepitope to so stimulate an enhanced immune response by providing local and/or systemic stimulation of immune reaction against the (infected) cells that produce and present the neoepitopes. Thus, contemplated compositions and methods will not only directly stimulate T-cell activation via neoepitope-associated stimulation/reduction of inhibition, but also indirectly stimulate an immune response against the neoepitopes via local and/or systemic administration of stimulatory and/or anti-inhibitory compositions (e.g., to so trigger release of further immune stimulating cytokines).

To trigger overexpression or transcription of stress signals, it is also contemplated that the patient may be treated with low-dose chemotherapy, preferably in a metronomic fashion, and/or low-dose radiation therapy. For example, it is generally preferred that such treatment will be effective to affect at least one of protein expression, cell division, and cell cycle, preferably to induce apoptosis or at least to induce or increase the expression of stress-related genes (and particularly NKG2D ligands). Thus, in one contemplated aspects, such treatment will include low dose treatment using one or more chemotherapeutic agents. Most typically, low dose treatments will be at exposures that are equal or less than 70%, equal or less than 50%, equal or less than 40%, equal or less than 30%, equal or less than 20%, equal or less than 10%, or equal or less than 5% of the LD₅₀ or IC₅₀ for the chemotherapeutic agent. Additionally, where advantageous, such low-dose regimen may be performed in a metronomic manner as described, for example, in U.S. Pat. Nos. 7,758,891, 7,771,751, 7,780,984, 7,981,445, and 8,034,375.

With respect to the particular drug used in such low-dose regimen, it is contemplated that all chemotherapeutic agents are deemed suitable. Among other suitable drugs, kinase inhibitors, receptor agonists and antagonists, anti-metabolic, cytostatic and cytotoxic drugs are all contemplated herein. However, particularly preferred agents include those identified to interfere or inhibit a component of a pathway that drives growth or development of the tumor. Suitable drugs can be identified using pathway analysis on omics data as described in, for example, WO 2011/139345 and WO 2013/062505. Most notably, so achieved expression of stress-related genes in the tumor cells will result in surface presentation of NKG2D, NKP30, NKP44, and/or NKP46 ligands, which in turn activate NK cells to specifically destroy the tumor cells. Thus, it should be appreciated that low-dose chemotherapy may be employed as a trigger in tumor cells to express and display stress related proteins, which in turn will trigger NK-cell activation and/or NK-cell mediated tumor cell killing. Additionally, NK-cell mediated killing will be associated with release of intracellular tumor specific antigens, which is thought to further enhance the immune response.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A recombinant nucleic acid vector, comprising:
a viral genome comprising a recombinant sequence portion encoding a plurality of genes, wherein the recombinant sequence portion is operably coupled to a regulatory sequence to allow for expression of the plurality of genes; and
wherein the plurality of genes encode four distinct stimulatory molecules and an inhibitory ligand for an immune checkpoint receptor; and
wherein the viral genome has at least one mutated or deleted protein coding sequence to reduce immunogenicity of a virus encoded by the viral genome.

2. The recombinant nucleic acid vector of claim 1 wherein at least one of the four distinct stimulatory molecules is selected form the group consisting of CD80 (B7.1), CD86 (B7.2), CD54 (ICAM-1/BB2), and CD11 (LFA-1).

3. The recombinant nucleic acid vector of claim 1 wherein at least two of the four distinct stimulatory molecules is selected form the group consisting of CD80 (B7.1), CD86 (B7.2), CD54 (ICAM-1/BB2), and CD11 (LFA-1).

4. The recombinant nucleic acid vector of claim 1 wherein at least three of the four distinct stimulatory molecules is selected form the group consisting of CD80 (B7.1), CD86 (B7.2), CD54 (ICAM-1/BB2), and CD11 (LFA-1).

5. The recombinant nucleic acid vector of claim 1 wherein the four distinct stimulatory molecules are CD80 (B7.1), CD86 (B7.2), CD54 (ICAM-1/BB2), and CD11 (LFA-1).

6-13. (canceled)

14. The recombinant nucleic acid vector of claim 1 wherein the immune checkpoint receptor is CTLA-4 or PD-1, and optionally wherein the inhibitory ligand comprises a transmembrane domain that anchors the ligand to a cell membrane.

15. The recombinant nucleic acid vector of claim 1 wherein the recombinant sequence portion further comprises a human cancer-associated sequence.

16. The recombinant nucleic acid vector of claim 15 wherein the human cancer-associated sequence further comprises a trafficking sequence that preferentially directs a gene product encoded by the cancer-associated sequence to a cytoplasmic compartment of a cell hosting the recombinant nucleic acid vector.

17. The recombinant nucleic acid vector of claim 15 wherein the human cancer-associated sequence further comprises a trafficking sequence that preferentially directs a gene product encoded by the cancer-associated sequence to a lysosomal or endosomal compartment of a cell hosting the recombinant nucleic acid vector.

18. The recombinant nucleic acid vector of claim 15 wherein the human cancer-associated sequence encodes a protein selected from the group consisting of a cancer associated antigen, a cancer specific antigen, and a patient- and tumor-specific neoantigen.

19. The recombinant nucleic acid vector of claim 1 wherein the virus is an adenovirus.

20. The recombinant nucleic acid vector of claim 19 wherein the at least one mutated or deleted protein coding sequence is selected from the group consisting of E1, E2b, and E3.

21. The recombinant nucleic acid vector of claim 1 wherein the virus is replication deficient.

22-24. (canceled)

25. A virus comprising the recombinant nucleic acid vector of claim 1.

26. The virus of claim 25 wherein the virus is a recombination deficient adenovirus lacking the E2b gene.

27. The virus of claim 26 wherein the four distinct stimulatory molecules are CD80 (B7.1), CD86 (B7.2), CD54 (ICAM-1/BB2), and CD11 (LFA-1), wherein the immune checkpoint receptor is CTLA-4, and wherein the recombinant sequence portion further comprises a human cancer-associated sequence.

28-29. (canceled)

30. A method of stimulating an immune response in a mammal in need thereof, comprising a step of administering a virus according to claim 25 under a protocol effective to stimulate the immune response.

31. The method of claim 30 wherein the step of administering is performed by subcutaneous or subdermal injection.

32. The method of claim 30 further comprising administering a low-dose chemotherapy or a low-dose radiation therapy to the mammal.

33. The method of claim 32 wherein the low-dose chemotherapy or the low-dose radiation therapy is metronomically administered.
